(19) United States
(12) Patent Application Publication
(10) Pub. No.: US 2004/0175712 A1
(43) Pub. Date: Sep. 9, 2004

(54) Method for the identification of activators of g protein-coupled receptors and nucleic acids encoding those receptors

(76) Inventors: Frederick S Hagen, Seattle, WA (US); Theo deVos, Seattle, WA (US); Pieter J Oort, Shoreline, WA (US); Brett Russell, Seattle, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/484,272

(22) PCT Filed: Jul. 18, 2002
(86) PCT No.: PCT/US02/23374

Related U.S. Application Data
(60) Provisional application No. 60/306,902, filed on Jul. 19, 2001.

(51) Int. Cl. C12Q 1/68

(52) U.S. Cl. 435/6

(57) ABSTRACT

The present invention provides methods for identifying activators of G protein-coupled receptors (GPCRs) and for identifying nucleic acids encoding the receptor(s) for each identified activator. The present invention is directed at the generation of GPCR-encoding nucleic acid pools for expression in oocytes, the generation of compound pools, and the multiplex screening of both the compound and nucleic acid pools. Through successive subdivision of both the compound and nucleic pools into subpools, both activators and GPCR nucleic acids are identified from complex compound and receptor repertoires.

### CROSS-REFERENCE TO RELATED APPLICATIONS

This application claims the benefit of U.S. patent application Ser. No. 60/306,902, filed Jul. 19, 2001, the disclosure of which is incorporated herein by reference.

### BACKGROUND OF THE INVENTION

G protein-coupled receptors (GPCRs) are the largest and most diverse group of transmembrane proteins involved in signal transduction. (Howard et al., Trends Pharmacol. Sci. 22:132-40, 2001.) GPCRs, a superfamily of seven transmembrane cell surface receptors, are involved in diverse cellular functions within an organism that include, for example, embryogenesis, neurotransmitter release, neurosensation (e.g., chemosensory functions such as taste and smell) (Mombaerts, Science 286:707-711, 1999), neuronal axon pathfinding (Mombaerts et al., Cell 87:675, 1996; Mombaerts et al., Cold Spring Harbor Symp. Quant. Biol. 56:135, 1996), leukocyte targeting to sites of inflammation (Tager et al., J. Exp. Med., 192:439-46, 2000), and cell survival, proliferation, and differentiation. (Ryan et al, J. Biol. Chem. 273:13613-24, 1998). The complexity of the GPCR repertoire surpasses that of the immunoglobulin and T cell receptor genes combined, with members of the GPCR superfamily estimated at as many as 2,000, or more than 1.5% of the human genome. Further, members of the GPCR superfamily are the direct or indirect target of more than 50% of the current pharmaceutical drugs used clinically in humans.

The responses of GPCRs to diverse stimuli, such as, for example, hormones, neurotransmitters, odorants, and light, are mediated through their selective association with and activation of intracellular, heterotrimeric guanine nucleotide-binding (G) proteins. Normally, upon binding of a specific extracellular ligand, the GPCR activates a G protein, which then activates one or more effector proteins. Alternatively, the ligand-free GPCR can be in an activated state, constitutively initiating G protein-mediated activation of effectors and with ligand binding inducing a shift of the GPCR to an inactive conformation. (Gershengorn et al., U.S. Pat. No. 6,087,115, 2000.) Typically, these effectors are either ion channels or enzymes that alter the concentrations of second messenger molecules.

While the human genome project has allowed for the identification of many genes encoding novel GPCRs through bioinformatics and the common characteristics of GPCR sequences, the biological functions of and ligands for these GPCRs remain largely unknown. Because GPCRs are positioned in the plasma membrane and are capable of initiating a wide variety of cellular responses to diverse extracellular mediators, GPCRs for which an active ligand has not yet been identified (orphan GPCRs) are likely to provide a path to discovering new cellular pathways or molecules that are important in human physiology. This potential for orphan GPCRs is evidenced by the successful development of many marketed therapeutic agents through modulation of GPCR function. Thus, the “de-orphaning” of these GPCRs has become an important focus of research in the industry.

One system that has enabled the study of many GPCRs utilizes the expression of the receptors in Xenopus oocytes. The coupling of many GPCRs to ion channels allows the activation or inhibition of these GPCRs to be monitored in oocytes via voltage clamping techniques. Heterologous GPCRs can be functionally expressed in the oocyte by injecting exogenous, GPCR-encoding mRNA into the oocyte and then allowing the oocyte's endogenous cellular machinery to translate and insert the receptors into the plasma membrane. (See, e.g., Houamed et al., Science 252:1318-21, 1991; Dahmen et al., J. Neurochem. 58:1176-79, 1992.) Following functional expression of receptors, the ability of ligands to induce transmembrane conductance changes can be observed via a two-electrode voltage clamp system (Dahmen et al., supra), which can detect either a depolarization or hyperpolarization of the membrane potential.

While the Xenopus system has provided a means for monitoring GPCR activation, current approaches do not allow for the efficient screening of diverse GPCR receptor-ligand interactions, thereby allowing the rapid identification of GPCR ligands for a wide spectrum of orphan GPCRs. Methods aimed at the identification of unknown GPCRs typically utilize known ligands to generate a cellular response. For example, a GPCR glutamate receptor has been cloned from rat brain by injecting oocytes with large pools of rat brain mRNA, followed by screening for activation only against known ligands (e.g., glutamate) with the successive subdivision of mRNA pools conferring ligand-responsiveness to the oocyte. (See Houamed et al., supra.) Because only a single or, at most, a few known ligands are used in the screen, the scope of GPCR-ligand interactions that can be identified is narrow.

Similarly, methods aimed at the identification of ligands or other modulators for known receptors or ion channels utilize only a single known receptor or ion channel in screens. For example, methods have been described for the identification of “negative antagonists” to a known, constitutively activated GPCR through co-expression of the GPCR with a reporter protein. (Gershengom et al., U.S. Pat. No. 6,087,115, 2000.) Also, methods have been described for the identification of modulators of known G protein activated potassium channels (Kir3.0 channels) utilizing host cells (e.g., oocytes) expressing functional Kir3.0 channels. (Lester et al., U.S. Pat. No. 5,744,324, 1998.)

Using similar methods in oocytes, researchers have identified ligands to specific, previously identified GPCRs. For example, a previously unknown ligand to a known human olfactory receptor protein (OR17-40) has been identified by the functional expression of OR17-40 in oocytes in the absence of other heterologous receptors, followed by activation of this receptor with a mixture of 100 different odorants; through subsequent subdivision of this mixture into progressively smaller groups, a single activating ligand has been identified. (See Wetzel et al., J. Neurosci. 19:7426-33, 1999.) Similarly, a ligand to a novel GPCR (GRL106) from the mollusc, Lymnaea stagnalis, has been identified by application of peptide extracts from the Lymanaea brain to Xenopus oocytes injected only with the GRL106-encoding cRNA and assaying for activation of a calcium-dependent chloride channel. (See Tensen et al., J. Neurosci. 18:9812-21, 1998.) In addition, a peptide ligand of a novel GPCR from Drosophila melanogaster has been identified by injecting only the Drosophila receptor-encoding mRNA into oocytes followed by application of Drosophila head extracts. (Birgul et al., EMBO J. 18:5892-900, 1999.)

However, all of these current approaches suffer from the disadvantage that either the GPCR or its ligand must be previously identified and, typically, functionally characterized to some degree. Consequently, screens have not been performed where large, complex arrays of GPCR-ligand interactions are involved, such as where large numbers of both orphan GPCRs and candidate ligands are used. In addition, the use of large number of compounds has been attempted due to concerns about high backgrounds obscuring signals.

Thus, due to the structural and functional complexity of the GPCR superfamily, current methods do not permit the efficient identification of ligands to GPCRs that have not been previously identified or characterized. There remains a need in the art for methods directed at the screening of diverse GPCR-ligand interactions and that, in the absence of an identified or functionally characterized GPCR or ligand, permit the identification of both a GPCR ligand and its corresponding receptor. Such methods are necessary to allow for the rapid identification of ligands for a wide spectrum of orphan GPCRs, thereby facilitating the determination of the GPCR function in physiology. The present invention provides such methods which are further set forth herein.

### SUMMARY OF THE INVENTION

The present invention generally relates to methods for identifying an RNA that encodes a G protein-coupled receptor (GPCR) of unknown function in a library of RNAs and for identifying an activator compound for the GPCR. In one aspect, the method includes simultaneously screening and subdividing a library of RNAs in oocytes and a library of compounds to identify the RNA that encodes the GPCR of unknown function and to identify the activator compound that causes a GPCR-mediated response. The activator compound causes the GPCR-mediated response when contacted with the oocytes expressing the RNA that encodes the GPCR of unknown function. In certain embodiments, the RNA library and/or the compound library can be complex.

The GPCR-mediated response can be, for example, an electrophysiological response. The GPCR-mediated response can be an increase or decrease in membrane potential. The electrophysiological response can be mediated through an endogenous oocyte G protein. Alternatively, a heterologous G protein or heterologous G protein subunit can be introduced into the oocytes to effect the GPCR-mediated response.

In one embodiment, the compounds in the compound library are affinity labeled. In another embodiment, the RNA library can include poly (A)+ mRNAs isolated from human cells or tissues. Alternatively, the RNA library can include RNAs transcribed from cDNAs. An RNA library also can be prepared using subtractive procedures, such as, for example, subtractive hybridization.

Other methods for identifying a G protein-coupled receptor (GPCR) of unknown function and an activator compound of the GPCR are also provided. Another method includes introducing a heterogeneous RNA pool into oocytes, an RNA in the RNA pool encoding the GPCR of unknown function. The oocytes are contacted with a plurality of pools of compounds. An activator compound pool is identified that causes a GPCR-mediated electrophysiological response when contacted with the oocytes expressing the pool of heterogeneous RNAs. The activator compound pool is subdivided into compound subpools, and the RNA pool is subdivided into RNA subpools. The RNA subpools are introduced into oocytes, which are contacted with the compound. An activator compound subpool is identified from the compound subpools, and a GPCR RNA subpool is identified, the activator compound subpool causing the GPCR-mediated electrophysiological response when contacted with an oocyte expressing the GPCR RNA subpool.

In certain embodiments, the RNA pool and/or the compound pools are complex. The GPCR can effect the electrophysiological response through an endogenous oocyte G protein. Alternatively, a heterologous G protein, or heterologous G protein subunit, can be introduced into the oocytes, the G protein or G protein subunit effecting the GPCR-mediated electrophysiological response. The electrophysiological response can be an increase or decrease in membrane potential.

In other embodiments, the steps of the method can be repeated, as necessary, to further subdivide the activator compound subpool and GPCR RNA subpool to identify an activator compound that induces the GPCR-mediated response and to identify a nucleic acid encoding the GPCR activated by the activator compound. The GPCR encoded by the RNA can be wild-type, a mutant GPCR associated with a disease, and the like.

The compound pools can include compound structures that overlap with compound structures of another compound pool. Alternatively, compound pools can include compound structures that are nonoverlapping with compound structures of other compound pools. The compounds in the compound pools optionally can be affinity labeled. Suitable affinity labels include, for example, FLAG, V5, myc, biotin, or polyhistidine.

The RNA pool can be poly (A)+ mRNAs isolated from human cells or tissues, RNAs transcribed from cDNAs, or other RNA's that include a RNA encoding a GPCR of unknown function. The RNA encoding the GPCR (of unknown function) can also be identified from a genomic database, such as, for example, by screening for nucleotide sequences that are substantially similar to a known GPCR. The RNA pool also can be prepared using subtractive procedures, such as, for example, subtractive hybridization.

In another aspect, a method is provided for producing a detectable electrophysiological response in an oocyte that is substantially characteristic of activation through a single, homogeneous type of G protein-coupled receptor (GPCR). The method generally includes expressing a plurality of different GPCRs of unknown function on an oocyte cell surface, contacting the oocyte with pools of compounds, and identifying the electrophysiological response.

In yet another aspect, a method is provided for identifying a G protein-coupled receptor (GPCR) of unknown function and an activator compound using multiple RNA pools. The method generally includes providing heterogenous pools of RNAs, at least one of the RNA pools including a RNA encoding the GPCR of unknown function. Pools of compounds are also provided. The RNA pools are introduced into oocytes to express the RNAs. The oocytes are then contacted with compound pools to identify an activator compound pool and a GPCR RNA pool. The activator compound pool causes a GPCR-mediated electrophysiological response when contacted with the oocytes expressing the GPCR RNA pool.

The activator compound pool is subdivided to form compound subpools, and the GPCR RNA pool is subdivided to form RNA subpools. The compound subpools are contacted with oocytes expressing the RNA subpools to identify the RNA encoding the GPCR and to identify the activator compound that causes the GPCR-mediated electrophysiological response when the activator compound is contacted with an oocyte expressing the RNA.

In a related aspect, a method is provided for identifying a G protein-coupled receptor (GPCR) of unknown function and an activator compound by comparing GPCR-mediated responses using different RNA pools. The method generally includes introducing a pool of heterogeneous RNAs from normal cells or tissues into first oocytes and introducing a pool of heterogeneous RNAs from cells or tissues having an altered GPCR-phenotype into second oocytes. The first and second oocytes are contacted with a plurality of pools of compounds. The GPCR-mediated electrophysiological responses in the first and second oocytes is compared to identify a difference in the electrophysiological response between the first and second oocytes.

Based on this difference, an activator compound pool and a GPCR RNA pool are identified, the activator compound pool causing the GPCR-mediated electrophysiological response when contacted to the second oocytes expressing the GPCR RNA pool. The activator compound pool is subdivided into compound subpools, and the GPCR RNA pool is subdivided into RNA subpools. The compound subpools are contacted with third oocytes expressing the RNA subpools to identify an activator compound subpool from the compound subpools, and to identify a GPCR RNA subpool. The activator compound subpool causes the GPCR-mediated electrophysiological response when contacted with an oocyte expressing the GPCR RNA subpool.

The steps of the method can be repeated, as necessary, to further subdivide the activator compound subpool and the GPCR RNA subpool to identify an activator compound that induces the electrophysiological response and to identify a nucleic acid encoding the GPCR activated by the activator compound. In an embodiment, the cells or tissues having an altered GPCR phenotype express a mutant GPCR of unknown function.

In another embodiment, a method is provided for identifying a G protein-coupled receptor (GPCR) of unknown function and an activator peptide. The method generally includes introducing a heterogeneous RNA pool into oocytes; an RNA in the RNA pool encodes the GPCR of unknown function. The oocytes are contacted with a plurality of pools of random or semi-random peptides to identify an activator peptide pool that causes a GPCR-mediated electrophysiological response when contacted with the oocytes expressing the pool of heterogeneous RNAs.

The activator peptide pool is subdivided into peptide subpools, and the RNA pool is subdivided into RNA subpools. The peptide subpools are contacted with oocytes expressing the RNA subpools to identify an activator peptide subpool from the peptide subpools, and to identify a GPCR RNA subpool. The activator peptide subpool causes a GPCR-mediated electrophysiological response when contacted with an oocyte expressing the GPCR RNA subpool. The RNA pool and/or the peptide pools can be complex.

The electrophysiological response can be, for example, an increase or decrease in membrane potential. In certain embodiments, the GPCR can effect the electrophysiological response through an endogenous oocyte G protein. Alternatively, a heterologous G protein, or heterologous G protein subunit, can be introduced into the oocytes, the G protein or G protein subunit effecting the GPCR-mediated electrophysiological response.

Optionally, the steps of the method can be repeated to further subdivide the activator peptide subpool and GPCR RNA subpool to identify an activator peptide that induces the electrophysiological response and to identify a nucleic acid encoding the GPCR activated by the activator peptide.

The GPCR can be, for example, wild-type, a mutant GPCR associated with a disease, and the like. The peptide subpools can comprise peptide sequences that overlap with peptide sequences of another peptide subpool or nonoverlapping peptide sequences. The peptides in the peptide pools optionally can be affinity labeled. Suitable affinity labels include, for example, FLAG, V5, myc, biotin, or polyhistidine.

The RNA pools can include, for example, poly (A)⁺ mRNAs isolated from human cells or tissues, RNAs transcribed from cDNAs, and the like. The RNA encoding the GPCR can also be identified from a genomic database. For example, a RNA encoding a GPCR of unknown function can be identified by screening for nucleotide sequences that are substantially similar to a known GPCR. The RNA pool also can be prepared using subtractive procedures, such as, for example, subtractive hybridization.

In a related aspect, a method is provided for producing a detectable electrophysiological response in an oocyte that is substantially characteristic of activation through a single, homogeneous type of G protein-coupled receptor (GPCR). The method generally includes expressing a plurality of different GPCRs of unknown function on an oocyte cell surface; contacting the oocyte with pools of random or semi-random peptides; and identifying the electrophysiological response.

In yet another aspect, a method for identifying a G protein-coupled receptor (GPCR) of unknown function and an activator peptide is provided. The method generally includes providing heterogenous pools of RNAs, at least one of the RNA pools including a RNA encoding the GPCR of unknown function. Pools of random or semi-random peptides are also provided. The RNA pools are introduced into oocytes to express the RNAs. The oocytes are contacted with the peptide pools to identify an activator peptide pool and a GPCR RNA pool; the activator peptide pool causing a GPCR-mediated electrophysiological response when contacted with the oocytes expressing the GPCR RNA pool.

The activator peptide pool is subdivided to form peptide subpools and the GPCR RNA pool is subdivided to form RNA subpools. The peptide subpools are contacted with oocytes expressing the RNA subpools to identify the RNA encoding the GPCR and to identify the activator peptide that causes the GPCR-mediated electrophysiological response when the activator peptide is contacted with an oocyte expressing the RNA.

In still another aspect, a method is provided for identifying a G protein-coupled receptor (GPCR) of unknown function and an activator by comparing the GPCR-mediated response of oocytes containing different RNA pools. The method generally includes introducing a pool of heterogeneous RNAs from normal cells or tissues into first oocytes, and introducing a pool of heterogeneous RNAs from cells or tissues having an altered GPCR phenotype into second oocytes. The first and second oocytes are contacted with a plurality of pools of random or semi-random peptides. The GPCR-mediated electrophysiological responses of the first and second oocytes is compared to identify a difference in the electrophysiological response between the first and second oocytes.

An activator peptide pool and a GPCR RNA pool are identified, the GPCR RNA pool being a pool from the cells or tissues having an altered GPCR phenotype. The activator peptide pool causes the GPCR-mediated electrophysiological response when contacted to the second oocytes expressing the GPCR RNA pool. The activator peptide pool is subdivided into peptide subpools and the GPCR RNA pool is subdivided into RNA subpools. The peptide subpools are contacted with third oocytes expressing the RNA subpools to identify an activator peptide subpool from the peptide subpools, and to identify a GPCR RNA subpool. The activator peptide subpool causes the GPCR-mediated electrophysiological response when contacted with an oocyte expressing the GPCR RNA subpool.

The steps of the method can optionally be repeated to further subdivide the activator peptide subpool and GPCR RNA subpool to identify an activator peptide that induces the electrophysiological response and to identify a nucleic acid encoding the GPCR activated by the activator peptide. In one embodiment, the cells or tissues having an altered GPCR phenotype express a mutant GPCR of unknown function.

A further understanding of the nature and advantages of the invention will become apparent by reference to the remaining portions of the specification.

### BRIEF DESCRIPTION OF THE DRAWING

FIG. 1 depicts a plasmid map for an oocyte expression vector. cDNA's of interest were cloned into a multiple cloning site flanked by 5′ and 3′ untranslated regions of the Xenopus globin gene. A T3 RNA polymerase primer site was located upstream of the 5′ globin UTR. In this example, the GPCR clone encoded the CCR3 gene. The vector is designed for RNA production by in vitro transcription/translation.

FIG. 2 depicts a plasmid map of the thioredoxin-random peptide fusion vector. Random oligonucleotides encoding random amino acid sequences were cloned in frame at the carboxy terminus of the thioredoxin gene in the peptide insertion site. The thioredoxin fusion consists of an amino terminal histidine tag sequence, the thioredoxin gene, a flag recognition element and the random peptide. The vector was designed for inducible expression in bacteria.

FIG. 3 depicts sample data from analysis of multiple receptors expressed in oocytes. Oocytes were injected with 14 GPCR receptors+GIRK 1+2, incubated for 5 days, and patch clamped in a perfusion chamber. Oocytes were exposed to 50 microliter random peptide library pools (pf838-pf846) each having an estimated diversity of 1000 unique peptides per pool. The oocyte was then perfused with sample buffer (20% hK) and 50 microliter samples of positive control ligands, dyno-(dynorphin) 100 nM, RANTES—(regulated upon activation, normal T cell expressed and secreted) 100 ng/ml, IL-8—(interleukin 8) 50 ng/ml, BLC—(B Lymphocyte Chemoattractant) 50 ng/ml. The peptide pools produced no detectable response, while perfusion with positive control libraries resulted in a −30 to −75 nA response.

### DESCRIPTION OF THE SPECIFIC EMBODIMENTS

The present invention provides methods for the parallel identification of both activators of GPCRs of unknown function and the nucleic acids encoding the activated GPCRs. The term “activator” means any compound that, when exposed to an oocyte expressing GPCRs, produces an electrophysiological response in the oocyte. An activator can evoke an electrophysiological response by agonistically or antagonistically modulating the activity of a G protein-coupled receptor. “Activation” of GPCRs means any effect on GPCR function that produces an electrophysiological response in the oocyte. Typically, activation of GPCRs will include, for example, a change in the association of a GPCR with a G-protein subunit(s), including any change in the affinity of G-protein βγ subunits for G protein α subunits.

The invention provides methods for expressing pools of GPCRs of unknown function in oocytes, the generation of large, complex pools of compounds, and the screening of these compound pools by exposing them to the oocytes expressing pools of GPCRs. Compound pools are screened for activation of a GPCR by detecting an electrophysiological response in the oocyte. The identity of an activator is determined by successive subdivision of activating compound pools into subpools until there is a one to one correspondence between a single compound and activation. Similarly, the GPCR activated by a specific compound is identified by successive subdivisions of GPCR-encoding nucleic acids until a nucleic acid conferring compound-responsiveness to the oocyte is identified. The present invention thus allows for the screening of a large number of diverse GPCR-compound interactions and the identification of both an activator of GPCRs and the corresponding activated GPCR of unknown function from complex compound and receptor repertoires.

The term “compound” means molecules that are potentially capable of structurally interacting with GPCRs through non-covalent interactions, such as, for example, through hydrogen bonds, ionic bonds, van der Waals attractions, or hydrophobic interactions. For example, compounds will most typically include molecules with functional groups necessary for structural interaction with proteins, glycoproteins, and/or other macromolecules, particularly those groups involved in hydrogen bonding.

Compounds can include small organic molecules such as, for example, aliphatic carbon or cyclical carbon (e.g., heterocyclic or carbocyclic structures and/or aromatic or polyaromatic structures). These structures can be substituted with one or more functional groups such as, for example, an amine, carbonyl, hydroxyl, or carboxyl group. In addition, these structures can include other substituents such as, for example, hydrocarbons (e.g., aliphatic, alicyclic, aromatic, and the like), nonhydrocarbon radicals (e.g., halo, alkoxy, acetyl, carbonyl, merapto, sulfoxy, nitro, amide, and the like), or hetero substituents (e.g., those containing non-carbon atoms such as, for example, sulfur, oxygen, or nitrogen).

Compounds can also include biomolecules. “Biomolecules” refer to classes of molecules that exist in and/or can be produced by living systems as well as structures derived from such molecules. Biomolecules typically include, for example, proteins, peptides, saccharides, fatty acids, steroids, purines, pyrimidines, and derivatives, structural analogs, or combinations thereof. Biomolecules can include one or more functional groups such as, for example, an amine, carbonyl, hydroxyl, or carboxyl group.

Compounds include those synthetically or biologically produced and can include recombinantly produced structures such as, for example, peptide-presenting fusion proteins. The term “fusion protein” refers to a polymer of amino acids produced by recombinant combination of two or more sequence motifs and does not refer to a specific length of the product; thus, a fusion protein can include a peptide sequence joined to an affinity label such as, for example, 6-histidine.

Preparation of Pools of GPCR-encoding Nucleic Acids

In one aspect of the invention, heterogeneous pools of nucleic acids encoding GPCRs of unknown function are prepared. The term “GPCR of unknown function” refers to GPCRs for which a natural ligand has not been identified and/or for which a function has not otherwise been identified. GPCRs of unknown function can include GPCRs from any organism.

The term “heterogeneous pools of nucleic acids” refers to any group of nucleic acids that includes a large plurality of different nucleic acid sequences and that includes, but is not necessarily limited to, a GPCR-encoding nucleic acid sequence. The term “large plurality” means any number of at least about 10 (e.g., about 20, about 30, about 40, or more). Heterogeneous pools of nucleic acids can also be complex. “Complex heterogeneous pool of nucleic acids” means a nucleic acid pool that includes about 50 or more different nucleic acid sequences. Complex heterogeneous pools of nucleic acids can contain, for example, about 100, 1,000, 10,000, or 100,000 or more different nucleic acid sequences each, with 100 to 1,000 being typical and 10,000 to 100,000 being more typical.

The term “nucleic acid” refer to a polymer composed of a multiplicity of nucleotide units (ribonucleotide or deoxyribonucleotide or related structural variants) linked via phosphodiester bonds. A nucleic acid can be of substantially any length, typically from about six (6) nucleotides to about 10⁹ nucleotides or larger. Nucleic acids include RNA, mRNA, cRNA, cDNA, genomic DNA, synthetic forms, and mixed polymers, both sense and antisense strands, and can also be chemically or biochemically modified or can contain non-natural or derivatized nucleotide bases, as will be readily appreciated by the skilled artisan. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, and the like), charged linkages (e.g., phosphorothioates, phosphorodithioates, and the like), pendent moieties (e.g., polypeptides), intercalators (e.g., acridine, psoralen, and the like), chelators, alkylators, and modified linkages (e.g., alpha anomeric nucleic acids, and the like).

GPCRs encoded by nucleic acids can be, for example, wild-type or mutant GPCRs. The term “wild-type GPCR” refers to any GPCR genotype and/or phenotype that is characteristic of a majority of individuals within a species in the natural environment. The term “mutant GPCRs” refers to those GPCRs that are genetically different than wild-type GPCRs. Such differences can be caused, for example, by changes in nucleic acid sequences (including substitutions, deletions, and/or insertion of foreign sequences), changes in gene position, and/or gene duplication.

The GPCR-encoding nucleic acids within a nucleic acid pool can be based on a variety of factors such as, for example, sequence similarity to known GPCRs (see infra), a cellular mechanistic target (e.g., leukocyte targeting or axonal pathfinding) expression in particular target cells or tissues (e.g., disease cells or tissues), or a particular function of the GPCR (e.g., chemosensory functions such as taste and smell). For example, if taste or smell is the particular function of interest, GPCR sequences with similarity to known GPCR odorant receptors can be identified using the techniques described infra. Nucleic acid pools comprising these odorant receptor sequence can then be generated (see infra) and used to screen for a broad class of odorant receptor activators that could function, for example, as components in food or perfume to stimulate taste or smell. Similarly, to screen for activators that could function in inflammation, GPCR sequences with similarity to known GPCRs that function, for example, in leukocyte targeting can comprise the nucleic acids within a nucleic acid pool. Alternatively, for example, a particular cell or tissue type (e.g., tumor-involved tissue or other cells or tissues representative of a disease target) can be the basis for generating nucleic acid pools by isolating GPCR-encoding nucleic acids expressed in those tissues (see infra).

In one exemplary embodiment, the heterogeneous pool of nucleic acids is obtained by isolating RNA from a natural source such as, for example, cells or tissues. Such cells or tissues can be selected, for example, according to GPCR expression levels. The RNA isolated can be total RNA, poly (A)⁺ RNA, or RNA enriched in receptor genes by isolation from the ribosomal component of rough endoplasmic reticulum. Methods for the isolation of total or poly (A)⁺ RNA from cells or tissues are known in the art. (See, e.g., Sambrook et al., Molecular Cloning: A Laboratory Manual, 3rd ed., Cold Spring Harbor Laboratory Press, NY (2001); Ausubel et al., Current Protocols in Molecular Biology, 4th ed. John Wiley and Sons, New York (1999); which are incorporated by reference in their entirety.) Poly (A)⁺ RNA can be obtained from total RNA by oligo d(T) affinity purification. (See Sambrook et al.; Ausubel et al., supra.) Alternatively, poly (A)⁺ RNA from various tissues is available from commercial suppliers, including Clontech and InVitrogen. If desired, the pool size of RNA isolated using the above methods can varied by fractionation of RNA, such as, for example, by electrophoresis through agarose gels or sedimentation through sucrose gradients. (See, e.g., Sambrook et al.; Ausubel et al., supra.)

In another exemplary embodiment, a cDNA library can be prepared from the isolated RNA pools using molecular biology techniques known in the art. (See Sambrook et al., supra; Ausubel et al., supra.) Also, kits for the generation of cDNA are available from commercial suppliers such as, for example, InVitrogen (Copy Kit™ cDNA Synthesis Kit). The cDNA is cloned into a vector (e.g., pGEMHE or pBSMTX) in which the cDNA insert is flanked at the 5′ side by an RNA polymerase binding site to allow for transcription of message and a restriction endonuclease cleavage site at the 3′ flank to linearize the DNA sequence. From the initial cDNA library, an appropriate size heterogeneous pool of nucleic acids can be obtained. For example, the cDNA library can be plated on selective media plates (e.g., LB+ampicillin) to form pools of an appropriate size, such as, for example, 100, 1,000, 10,000, or 100,000 unique clones. (See, e.g., Sambrook et al.; Ausubel et al., supra.)

In a third exemplary embodiment, cDNA clones for GPCRs of unknown function can be isolated by standard molecular biology methods. In this approach, nucleic acid sequences for GPCRs of unknown function can be identified from a number of sources including, for example, publications or public or private databases. Name searches of the Genbank database for known GPCR sequences reveal hundreds of these receptors from numerous species. Name searches for the generic term GPCR also reveal numerous known receptors and receptors categorized as orphans. In addition organized GPCR sequence repositories such as, for example, the GPCRDB (http://www.gpcr.org/7tm/) contain organized lists of receptors. Specific receptor subclass information is annotated in specific databases such as the Olfactory Receptor Database (ORDB) (http://ycmi.med.yale.edu/senselab/ordb/).

Identification of additional receptors can be based on similarity to known GPCR sequences and by analysis of cDNA sequences for characteristic seven transmembrane domain regions. Sequence similarity can be determined, for example, by using available sequence comparison programs. (See discussion of sequence comparison methods, infra.). Using oligonucleotides based on the gene sequence of an identified GPCR-homologue or, alternatively, oligonucleotides based on conserved sequences determined by gene sequence alignment (see discussions of sequence alignment and oligonucleotide sequence selection, infra), the cDNA sequence is then cloned by standard methods known in the art using polymerase chain reaction (PCR) amplification. (See discussion of PCR methods, infra; PCR Applications: Protocols for Functional Genomics (Innis et al., eds., 1999), incorporated by reference in its entirety and hereinafter “PCR Applications.” See also Sambrook et al., supra; Ausubel et al., supra.) Alternatively, nucleic acid probes can be generated based on identified sequences for GPCRs of unknown function or, alternatively, on conserved sequence regions. These probes can be used to clone nucleic acids encoding GPCRs of unknown function from cDNA libraries. (See discussion of hybridization and expression cloning of GPCR cDNAs, infra.) The GPCR-encoding cDNAs are cloned into a vector containing a 5′ RNA polymerase binding site, and a 3′ endonuclease site as described above.

The term “oligonucleotide” refers to a nucleic acid of from about six (6) to about one hundred (100) nucleotides or to about one thousand (1000) nucleotides or more in length. Thus, oligonucleotides are a subset of nucleic acids. Oligonucleotides can be obtained, for example, by synthesis on an automated oligonucleotide synthesizer (e.g., those manufactured by Applied BioSystems (Foster City, Calif.)) according to specifications provided by the manufacturer.

In yet another exemplary embodiment, nucleic acid pools can be derived from cells or tissues having an altered GPCR phenotype. The term “altered GPCR phenotype” refers to any difference in the GPCRs expressed by a particular cell population or tissue from those GPCRs normally expressed by cells or tissues of the same type. Such differences can include, for example, expression of wild-type GPCRs not normally expressed, overexpression of GPCRs normally expressed at low levels, or expression of mutant GPCRs. Cells or tissues having an altered GPCR phenotype can include, for example, cells or tissues that are diseased or those treated with pharmacological agents.

Using the methods according to the present invention, both nucleic acids encoding GPCRs differentially expressed in cells or tissues having an altered GPCR phenotype and activators of these GPCRs can be identified. For example, the electrophysiological responses of oocytes expressing a nucleic acid pool from cells or tissues having an altered GPCR phenotype can be compared with those of oocytes expressing a nucleic acid pool from normal cells or tissues. Compound pools or subpools that exhibit differences in activation between these two series of oocytes can be successively subdivided (see infra) to identify the activators that cause the different electrophysiological responses. Nucleic acid pools conferring activator-responsiveness in oocytes can be successively subdivided (see infra) to identify nucleic acids from cells or tissues having an altered GPCR phenotype that confer the different electrophysiological responses in oocytes.

Alternatively, nucleic acid pools from cells or tissues having an altered GPCR phenotype can be prepared by subtractive procedures. The term “subtractive procedures” refers to any procedure in which nucleic acids from at least two different cell populations or tissues are compared and those nucleic acids differentially expressed by either cell population or tissue are selected and/or identified. For example, subtractive procedures can be performed by subtractive hybridization of nucleic acids. The term “subtractive hybridization” refers to any procedure in which a single-stranded nucleic acid derived from at least two different cell populations or tissues is hybridized and then single-stranded (non-hybridized) nucleic acids are separated from double-stranded (hybridized) nucleic acids. (See, e.g., Sambrook et al., supra; Ausubel et al., supra.) The term “subtractive library” refers to any nucleic acid library prepared by subtractive hybridization. Methods for preparing subtractive libraries are known in the art. (See Sambrook et al., supra; Ausubel et al., supra.) Subtractive libraries that include nucleic acids encoding differentially expressed GPCRs can be prepared, for example, by subtractive hybridization of nucleic acids (e.g., single-stranded cDNA) derived from cells or tissues having an altered GPCR phenotype with nucleic acids (e.g., mRNA) from normal cells or tissues. For example, cDNA derived from diseased tissue (e.g., tissue that is tumor-involved or inflamed) can be subtractively hybridized with mRNA from the same tissue (typically from the same donor) that is non-diseased (e.g., non-tumor-involved or non-inflamed). The cDNAs differentially expressed in the cells or tissue having the altered GPCR phenotype can then represent a nucleic acid pool to be expressed in oocytes or, alternatively, a library from which multiple nucleic acid pools can be generated.

Alternatively, subtractive procedures can be performed by comparing, for example, nucleic acid sequences of libraries from genomic databases. For example, subtractive procedures can be performed electronically using databases available through commercial sources (e.g., Human Genome Sciences, Inc. or Incyte, Inc.). Using software programs provided, for example, by the manufactures, nucleic acid sequences represented by an expression library of interest can be compared to any other nucleic acid expression library in the database and those sequences differentially represented in the library of interest identified. Using these identified sequences, a nucleic acid pool can be prepared using standard molecular biology techniques known in the art such as, for example, PCR amplification and subcloning of the identified sequences. (See, e.g., Sambrook et al., supra; Ausubel et al., supra; discussion of PCR methods, infra.)

In another exemplary embodiment of the invention, nucleic acids encoding promiscuous G proteins or G protein subunits can be co-injected with nucleic acid pools encoding GPCRs. The term “promiscuous G proteins or G protein subunits” refers to those G proteins or subunits that can associate with multiple types of GPCRs. Multiple types of GPCRs can include GPCRs that are structurally and/or functionally similar (e.g., GPCRs within a broad class such as odorant receptors) as well as GPCRs that are structurally and or functionally dissimilar (e.g., GPCRs from different classes such as odorant receptors and neurotransmitter receptors). Examples of promiscuous G proteins include, for example, G alpha 14 and G alpha 15. Nucleic acids encoding promiscuous G proteins can be prepared, for example, by PCR amplification and subcloning of G proteins sequences (e.g., Accession #: M80631 or M80632) from nucleic acid libraries, using techniques known in the art. (See discussion of PCR methods, infra. See also Sambrook et al., supra; Ausubel et al., supra.) To express promiscuous G proteins in oocytes, from about 0.01 ng to about 10 ng of promiscuous G protein-encoding RNAs can be co-injected with nucleic acid pools.

Similarly, in another exemplary embodiment, nucleic acids expressing specific ion channels can be co-injected with nucleic acid pools. Examples of ion channels that can be co-expressed include, for example, voltage-gated potassium channels (e.g., GIRK channels KIR 3.1-3.4) or the cystic fibrosis transmembrane-conductance regulator (CFTR). Nucleic acids encoding specific ion channels can be prepared, for example, by PCR amplification and subcloning of ion channel sequences (e.g., Accession Nos. D45022, U51122, or NM 000492) from nucleic acid libraries. To express ion channels in oocytes, from about 0.1 ng to about 10 ng of ion channel-encoding RNAs can be co-injected with nucleic acid pools.

GPCR Sequence Alignments and Comparisons: For sequence comparison, either nucleic acid or polypeptide sequences can be used. Typically, one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Prior to discussing sequence alignments and comparisons in more detail, it may be helpful to set forth definitions of certain terms. The term “polypeptide” refers to a polymer of amino acids and its equivalent and does not refer to a specific length of the product; thus, peptides, oligopeptides and proteins are included within the definition of a polypeptide.

The terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence, as measured using one of the following sequence comparison algorithms, or by visual inspection.

The phrase “substantially identical,” in the context of two nucleic acids or polypeptides, refers to two or more sequences or subsequences that have at least 60%, typically 80%, most typically 90-95% nucleotide or amino acid residue identity, when compared and aligned for maximum correspondence, as measured using one of the following sequence comparison algorithms, or by visual inspection. An indication that two polypeptide sequences are “substantially identical” is that one polypeptide is immunologically reactive with antibodies raised against the second polypeptide.

“Similarity” or “percent similarity” in the context of two or polypeptide sequences, refer to two or more sequences or subsequences that are the same or have a specified percentage of amino acid residues or conservative substitutions thereof, that are the same, when compared and aligned for maximum correspondence, as measured using one of the following sequence comparison algorithms, or by visual inspection. By way of example, a first amino acid sequence can be considered similar to a second amino acid sequence when the first amino acid sequence is at least 60%, 70%, 75%, 80%, 90%, or even 95% identical, or conservatively substituted, to the second amino acid sequence when compared to an equal number of amino acids as the number contained in the first sequence, or when compared to an alignment of polypeptides that has been aligned by a computer similarity program known in the art, as discussed below.

The term “substantial similarity” in the context of polypeptide sequences, indicates that the polypeptide comprises a sequence with at least 70% sequence identity to a reference sequence, or typically 80%, or more typically 85% sequence identity to the reference sequence, or most typically 90% identity over a comparison window of about 10-20 amino acid residues. In the context of amino acid sequences, “substantial similarity” further includes conservative substitutions of amino acids. Thus, a polypeptide is substantially similar to a second polypeptide, for example, where the two peptides differ only by one or more conservative substitutions.

Optimal alignment of sequences for comparison can be conducted, for example, by the local homology algorithm of Smith & Waterman (Adv. Appl. Math. 2:482, 1981, which is incorporated by reference herein), by the homology alignment algorithm of Needleman & Wunsch (J. Mol. Biol. 48:443-53, 1970, which is incorporated by reference herein), by the search for similarity method of Pearson & Lipman (Proc. Nat. Acad. Sci. USA 85:2444-48, 1988, which is incorporated by reference herein), by computerized implementations of these algorithms (e.g., GAP, BESTFIT, FASTA, and TFASTA in the Wisconsin Genetics Software Package, Genetics Computer Group, 575 Science Dr., Madison, Wis.), or by visual inspection. (See generally Ausubel et al., supra.) In addition, the Olfactory Receptor Database (http://ycmi.med.yale.edu/senselab/ordb/) provides tools and resources for accessing GPCR phylogenetic trees and alignments of GPCR sensory chemoreceptors. (See Skoufos et al., Nucleic Acid Res. 28:341-43, 2000, incorporated by reference in its entirety.)

One example of a useful algorithm for sequence comparisons is PILEUP. PILEUP creates a multiple sequence alignment from a group of related sequences using progressive, pairwise alignments to show the percent sequence identity. It also plots a tree or dendogram showing the clustering relationships used to create the alignment. PILEUP uses a simplification of the progressive alignment method of Feng and Doolittle (J. Mol. Evol. 25:351-60, 1987, which is incorporated by reference herein). The method used is similar to the method described by Higgins & Sharp (Comput. Appl. Biosci. 5:151-53, 1989, which is incorporated by reference herein). The program can align up to 300 sequences, each of a maximum length of 5,000 nucleotides or amino acids.

The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of an algorithm that is suitable for determining percent sequence identity and sequence similarity is the BLAST algorithm, which is described by Altschul et al. (J. Mol. Biol. 215:403-410, 1990, which is incorporated by reference herein). (See also Zhang et al., Nucleic Acid Res. 26:3986-90, 1998; Altschul et al., Nucleic Acid Res. 25:3389-402, 1997, which are incorporated by reference herein.) Software for performing BLAST analyses is publicly available through the National Center for Biotechnology Information (NCBI) (http://www.ncbi.nlm.nih.gov/). BLAST analysis can be performed using nucleic acid or protein sequences. For example, BLASTP (protein sequence) or BLASTX (nucleic acid sequence) searches can be performed with the NCBI BLAST network service to search databases including GenBank, SwissPro, PIR, and the Brookhaven Protein Data Bank. (See Altschul et al., supra.)

The BLAST algorithm involves first determining high scoring sequence pairs (HSPs) by determining short words of length W in the query sequence, which either match or satisfy some positive-valued threshold score T when aligned with a word of the same length in a database sequence. T is referred to as the neighborhood word score threshold (Altschul et al., 1990, supra). These initial neighborhood word hits act as seeds for initiating searches to find longer HSPs containing them. The word hits are then extended in both directions along each sequence for as far as the cumulative alignment score can be increased. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff & Henikoff, Proc. Nat. Acad. Sci. USA 89:10915-9, 1992, which is incorporated by reference herein) alignments (B) of 50, expectation (E) of 10, M=5, N=−4, and a comparison of both strands.

In addition to calculating percent sequence identity, the BLAST algorithm also performs a statistical analysis of the similarity between two sequences (see, e.g., Karlin & Altschul, Proc. Nat. Acad. Sci. USA 90:5873-77, 1993, which is incorporated by reference herein). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more typically less than about 0.01, and most typically less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. The term “immunological cross-reactive” means that a polypeptide, fragment, derivative or analog is capable of competitively inhibiting the binding of an antibody to its antigen.

PCR Amplification of GPCR-encoding Nucleic Acid Sequences: GPCR-encoding nucleic acid sequences can be isolated, for example, by polymerase chain reaction (PCR) to amplify GPCR sequences in a genomic or cDNA library. Oligonucleotide primers representing sequences of GPCRs of unknown function identified in databases or, alternatively, conserved regions of known GPCRs, as described above, can be used as primers in PCR. In a typical embodiment, the oligonucleotide primers represent at least a fragment of conserved segments of identity between GPCRs of different species. Synthetic oligonucleotides can be utilized as primers to amplify particular sequences within a GPCR gene from a source (e.g., RNA or DNA), typically a cDNA library or mRNA of potential interest. PCR can be carried out, for example, by use of a Perkin-Elmer Cetus thermal cycler and Taq polymerase (Gene Amp®). Degenerate primers for use in the PCR reactions can be synthesized. For example, the CODEHOP strategy of Rose et al. (Nucl. Acids Res. 26:1628-35, 1998, which is incorporated by reference herein) can be used to design degenerate PCR primers using multiply-aligned sequences as a reference. Methods for performing PCR and related methods are well known in the art. (See, e.g., U.S. Pat. Nos. 4,683,202, 4,683,195 and 4,800,159; Innis et al., PCR Protocols: A Guide to Methods and Applications, Academic Press, Inc., San Diego, Calif. (1989); PCR Applications, supra; White (ed.), PCR Cloning Protocols: From Molecular Cloning to Genetic Engineering, Humana Press, (1996); EP 320 308; which are incorporated by reference herein in their entirety.)

In one embodiment, degenerate primers are used to isolate human cDNAs encoding GPCRs of unknown function. Briefly, an alignment of multiple known GPCR polypeptide sequences from humans or, alternatively, from different species (including or not including human sequences; non-human species can include, for example, mouse, C. elegans, and Drosophila) is prepared and used to visually identify blocks of sequences having substantial similarity (e.g., 70%, 80%, or 90% sequence identity) and low codon degeneracy (see Rose et al., supra). The CODEHOP strategy is used to design degenerate primers based on the blocks of low codon degeneracy. Pools of primers varying in redundancy from 2 fold to about 32 fold are prepared. A hemi-nested PCR strategy is used to amplify fragments from a human cDNA library. Briefly, PCR is performed at or below Tₘ of degenerate mixture (e.g., 55° C.) using the primer pools. (See, e.g., Rose et al., supra; Rose et al., J Virology 71:4138-44, 1997.) PCR amplification products can be detected, for example, by agarose gel electrophoresis. The identity of the PCR amplification products can be confirmed by DNA sequence analysis. Once the identity of the PCR amplification products is confirmed the amplification products can be used to isolate full length GPCR cDNA from the human cDNA library. (See, e.g., Sambrook et al., supra; Ausubel et al., supra.)

Expression and Hybridization Cloning of GPCR cDNAs: For expression cloning (a technique commonly known in the art; see, e.g., Sambrook et al., supra; Ausubel et al., supra), an expression library is constructed by methods known in the art. For example, mRNA encoding a GPCR is isolated cDNA is prepared and then ligated into an expression vector (e.g., a bacteriophage derivative) such that the GPCR is capable of being expressed by the host cell into which it is then introduced. Various screening assays can then be used to select for the expressed GPCR polypeptides. For example, polyclonal antibodies against conserved polypeptide regions of an identified subfamily of GPCRs of unknown function can be used to screen a human cDNA expression library (e.g., from Strategene) to identify human cDNA clones within that subfamily.

Alternatively, GPCR-encoding nucleic acids can also be isolated by hybridization using a heterologous GPCR nucleic acid as a probe. (See, e.g., Sambrook et al., supra; Ausubel et al., supra.) For example, GPCR-encoding nucleic acids can be isolated by screening a cDNA library. A portion of a GPCR gene or its specific RNA, or a fragment thereof, that exhibits low codon degeneracy can be purified, labeled, and used to screen a library by nucleic acid hybridization. Hybridization procedures can be performed under low, moderate, or high stringency conditions. (See, e.g., Sambrook et al., supra; Ausubel et al., supra.) Those DNA fragments with substantial identity to the probe will hybridize.

Expression of RNAs in Oocytes

The heterogeneous nucleic acid pools prepared as described above, nucleic acid subpools prepared by subdivision of pools (see subdivision of nucleic acid pools, infra), or homogeneous nucleic acids isolated from pools or subpools (e.g., individual cDNA clones) generally are expressed in oocytes (e.g., Xenopus oocytes) following injection of RNAs. If the nucleic acids are poly (A)⁺ RNA or total RNA isolated from cells or tissues, the isolated RNA itself can be injected into the oocytes. If the nucleic acids are cDNAs, the pools, subpools, or individual clones are typically transcribed in vitro to produce transcripts (typically capped) prior to oocyte injection. The vector containing the GPCR cDNA sequence can be utilized as a template for RNA synthesis. Alternatively, if PCR is used to generate cDNAs, the PCR product can be used directly for RNA synthesis.

In Vitro Transcription: RNA for injection into oocytes can be prepared from nucleic acids using techniques known in the art. (See generally Sambrook et al., supra; Ausubel et al., supra.) RNA polymerases (e.g., T7, T3, or SP6 RNA polymerase) can be used to translate RNA transcripts in vitro from nucleic acid sequences downstream of specific promoters. These techniques can be performed, for example, according to the methods described in Sambrook et al. or Ausubel et al., supra. Alternatively, in vitro transcription of RNA can be performed using available commercial kits according to the manufacturer's instructions (e.g., the mMessage mMachine system available from Ambion, Inc.). Vectors containing the nucleic acid sequences to be transcribed can be linearized by endonuclease digestion prior to in vitro transcription. (See, e.g., Birgul et al., EMBO J. 18:5892-5900, 1999, incorporated herein by reference.) Alternatively, to permit in vitro transcription without endonuclease digestion, transcriptional terminators (e.g., in the case of T7 RNA polymerase, the bacteriophage T7 transcriptional terminator) can be included in the cloning vector used. (See, e.g., Mulvihill et al., U.S. Pat. No. 5,747,267, 1998, incorporated herein by reference.)

Oocyte Harvest: Techniques for harvesting and preparing oocytes used to express RNAs are known in the art. (See generally Dascal & Lotan (1992) in Methods in Molecular Biology, v. 13: Protocol in Molecular Neurobiology, eds. Longstaff & Revest. See also, e.g., Mulvihill et al., U.S. Pat. No. 5,747,267, 1998; Getchell et al., Neurochem. Res. 15:449-456, 1990; Getchell, Neurosci. Lett. 91:217-221, 1988.) As a typical example, adult female frogs (Xenopus laevis) are anesthetized, and then sections of ovaries surgically removed. Isolated oocytes are denuded of overlying follicle cells by, for example, agitation in 2 mg/ml collagenase (Sigma type IA) in: 82 mM NaCl, 2 mM KCl, 20 mM MgCl₂, 5.0 mM Na-HEPES (pH 7.5) for 1-2 hours. Oocytes of an appropriate stage, e.g., stage V and VI Xenopus oocytes, are then selected.

Oocyte Injection and Expression of RNAs: Techniques for cytoplasmic injections of RNA are known in the art. (See, e.g., Mulvihill et al., supra; Lindqvist, The Setup of a Two-electrode Voltage Clamp Technique for Xenopus Oocytes in Order to Study the L-type Voltage-dependent Ca²⁺ Channel, (2000) (Masters Thesis, Karolinska Institutet, Stockholm, Sweden) (on file at Karolinska Institutet and also available at http://www.d.kth.se/˜d99-pli/biomedicine/thesis.html), incorporated herein by reference in its entirety.) RNA injections are performed on denuded oocytes using, for example, a glass microelectrode and a NanoJect II injection system. Amounts of RNA injected can vary from about 0.01 to about 10 ng per oocyte with from about 1 up to about 10 ng of in vitro transcribed RNA being typical. RNA can be injected in volumes ranging from 10 to 100 nl of buffer, with about 50 nl of buffer representing a typical volume. Under different experimental conditions, injected RNA can include nucleic acids encoding GPCR's of interest, G-proteins and/or ion channels.

Oocytes injected with RNA are maintained at about 16 to about 19° C. in an appropriate buffer (e.g., ND96 (96 mM NaCl, 2 mM KCl, 1 mM MgCl₂, 5 mM Na-HEPES (pH 7.5) 1 mM CaCl₂) plus 2.5 mM Na-pyruvate, 50 μg/ml gentamicin, 5% horse serum). The oocytes are incubated for about 2 to about 5 days to allow for synthesis of protein and insertion of the GPCRs into the cell membrane of the oocytes.

Generation of Compound Pools for Identification of GPCR Activators

In one aspect of the invention, pools of compounds are generated. The term “pool of compounds” refers to mixture of compounds that substantially includes a large plurality of heterogeneous compound structures. The term “large plurality” means any number of compounds of at least about 10 (e.g., about 20, about 30, about 40, or more). Pools of compounds can also be complex. “Complex pool of compounds” means a compound pool that includes about 50 or more different compound structures. Complex pools of compounds can contain, for example, about 100, 1,000, 10,000, or 100,000 or more different compounds each, with 1,000 being typical and 10,000 to 100,000 being more typical.

Compound pools can be prepared from, for example, a historical collection of compounds synthesized in the course of pharmaceutical research; libraries of compound derivatives prepared by rational design (see generally, Cho et al., Pac. Symp. Biocompat. 305-16, 1998; Sun et al., J. Comput. Aided Mol. Des. 12:597-604, 1998; each incorporated herein by reference in their entirety), such as, for example, by combinatorial chemistry (see discussion of combinatorial chemical libraries, infra); natural products libraries (libraries including, for example, complex extracts derived from microorganisms such as bacteria, algae, fungi, yeasts, molds, and others; such libraries can, for example, include those formed in the course of pharmaceutical research); peptide libraries (see discussion of peptide libraries, infra); and the like.

In addition, a pool of compounds can consist of or be derived from a biological sample (e.g., tissue, cells, fluid, secretion, excretion, and the like) or chromatographic separation of such biological sample extracts using standard chromatographic techniques. In this case, if multiple pools are used, extracts from different biological samples (e.g., brain, heart, liver, spleen, etc.) can, for example, represent different compound pools. In addition, such biological samples can be prepared from diseased or disease-associated samples (e.g., tumors or tumor-involved tissue).

In one exemplary embodiment of the invention, compounds within a compound pool are affinity labeled. The term “affinity label” means any molecule capable of binding another molecule (a binding partner) with sufficient affinity and avidity to allow detection and/or some degree of purification based on the binding interaction. Affinity labels can include, for example, epitopes for antibody binding (e.g., FLAG, V5, or myc) or sequences that bind to non-antibody molecules (e.g., polyhistidine or biotin). The affinity label can be used to affinity purify the compounds. Methods for affinity purification (e.g., immunopurification using antibodies or purification of 6-histidine containing sequences by nickel or cobalt affinity chromatography) are known in the art. (See, e.g. Harlow & Lane, Antibodies, A Laboratory Manual, Cold Spring Harbor Laboratory Press, NY (1988); Piatibratov et al., Biochim. Biophys. Acta 1524:149-54, 2000; incorporated by reference herein in their entirety.)

Compound pools are used in screens for GPCR activation by contacting the pools with GPCR-expressing oocytes and recording any electrophysiological response (see discussion of screening for GPCR activation, infra).

Peptide Libraries: In one exemplary embodiment, compound pools can be prepared from peptide libraries. Generally, peptides ranging in size from about 4 amino acids to about 100 amino acids can be used, with peptides ranging from about 5 to about 20 being typical, with from about 5 to about 16 being more typical and from about 8 to about 16 being most typical.

In some aspects, the library can comprise synthetic peptides. For example, a population of synthetic peptides representing all possible amino acid sequences of length N (where N is a positive integer), or a subset of all possible sequences, can comprise the peptide library. Such peptides can be synthesized by standard chemical methods known in the art (see, e.g., Hunkapiller et al., Nature 310:105-11, 1984; Stewart and Young, Solid Phase Peptide Synthesis, 2^{nd} Ed., Pierce Chemical Co., Rockford, Ill., (1984)), such as, for example, an automated peptide synthesizer. Furthermore, if desired, nonclassical amino acids or chemical amino acid analogs can be used in substitution of or in addition into the classical amino acids. Non-classical amino acids include but are not limited to the D-isomers of the common amino acids, α-amino isobutyric acid, 4-aminobutyric acid, 2-amino butyric acid, γ-amino butyric acid, 6-amino hexanoic acid, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, selenocysteine, fluoro-amino acids, designer amino acids such as β-methyl amino acids, C α-methyl amino acids, N α-methyl amino acids, and amino acid analogs in general. Furthermore, the amino acid can be D (dextrorotary) or L (levorotary).

Peptide libraries can also be produced by transcription and translation from a library of nucleic acid sequences. For example, oligonucleotide libraries can be produced from fragments of genomic DNA and/or cDNA from a particular organism. Methods of making randomly sheared genomic DNA and/or cDNA, and of manipulating such DNAs, are known in the art. (See Sambrook et al., supra; Ausubel et al., supra.) Also, a random peptide library can be produced from a population of synthetic oligonucleotides encoding all possible amino acid sequences of length N (where N is a positive integer), or a subset of all possible sequences. Alternatively, a semi-random library can be used. For example, a semi-random library can be designed according to the codon usage preference of the host cell or to minimize the inclusion of translational stop codons in the encoded amino acid sequence. As an example of the latter, in the first position of each codon, equimolar amounts of C, A, and G and a one half-molar amount of T would be used. In the second position, A is used at a one half-molar amount while C, T, and G would be used in equimolar amounts. In the third position, only equimolar amounts of G and C would be used. Methods of making synthetic DNA are known to those of skill in the art. (See, e.g., Glick and Pasternak, Molecular Biotechnology: Principles and Applications of Recombinant DNA, ASM Press, Washington, D.C., 1998.)

Such oligonucleotides can optionally include any suitable cis regulatory sequence, such as, for example, a promoter, a ribosomal binding site, a translational start codon, a translational termination signal, a transcriptional termination signal, a polyadenylation signal, a cloning site (e.g., a restriction enzyme sites or cohesive end(s)), a sequence encoding an epitope, and/or a priming segment. For example, a library can include oligonucleotides having a restriction enzyme site near one end, operably associated with an ATG start codon, a random or semi-random sequence of N nucleotides, a translational stop codon, a primer binding site and a restriction enzyme site at the other end.

Such a collection of oligonucleotides can be directly ligated into a vector, into an expression vector (i.e., a vector that includes specific cis regulatory sequences in an expression cassette to effect expression of nucleic acid inserts; see infra), and the like. The oligonucleotides can be introduced into a vector as single stranded or double stranded DNA, and as either sense or antisense strands. As will be appreciated by the skilled artisan, double stranded nucleic acids can be formed, for example, by annealing complementary single stranded nucleic acids together or by annealing a complementary primer to the nucleic acid and then adding polymerase and nucleotides (e.g., deoxyribonucleotide or ribonucleotide triphosphates) to form double stranded nucleic acids. Double stranded nucleic acids can also be formed by ligating single stranded nucleic acids (e.g., DNA) into a site with 5′ and 3′ overhanging ends and then filling in the partially single stranded nucleic acids with a polymerase and nucleotide triphosphates.

In an exemplary embodiment, a library is created according to the following procedure using methods that are well known in the art. (See, e.g., Ausubel et al., supra; Sambrook et al., supra.) Double stranded DNA fragments are prepared from random or semi-random synthetic oligonucleotides, randomly cleaved genomic DNA and/or randomly cleaved cDNA. These fragments are treated with enzymes, as necessary, to repair their ends and/or to form ends that are compatible with a cloning site in an expression vector. The DNA fragments are then ligated into the cloning site of copies of the expression vector to form an expression library. The expression library is introduced into a suitable host strain, such as an E. coli strain, and clones are selected. The number of individual clones is typically sufficient to achieve reasonable coverage of the possible permutations of the starting material. The clones are combined and grown in mass culture, or in pools, for isolation of the resident vectors and their inserts. This process allows large quantities of the expression library to be obtained in preparation for subsequent procedures described herein. The details of manipulating and cloning oligonucleotides are known in the art, as well as the details of library construction, manipulation and maintenance. (See, e.g., Ausubel et al., supra; Sambrook et al., supra.)

Expression cassettes and/or vectors are used to express peptides encoded by sequences of an expression library. There are numerous expression cassettes and vectors known in the art which are readily available for use. (See, e.g., Ausubel et al., supra; Sambrook et al., supra.) To effect expression of peptides, an expression cassette can include, for example, in a 5′ to 3′ direction relative to the direction of transcription, a promoter region operably associated with a cloning site for insertion of library sequences and a transcriptional termination region, optionally including a polyadenylation (poly A) sequence. The expression cassette can optionally include a ribosome binding sequence, a translation initiation codon, and/or a translational termination codon. A secretion signal can be included adjacent the cloning site. Suitable secretion signals include, for example, the CD24 or IL-3 receptor secretory signals. The details of expressing nucleic acid libraries in host cells are known in the art. (See, e.g., Ausubel et al., supra; Sambrook et al., supra.)

In one exemplary embodiment, peptide libraries are produced by transcription and translation of a library of nucleic acid sequences cloned as a fusion to an affinity label or to a scaffold protein containing the affinity label. Suitable affinity labels include, for example, FLAG, V5, myc, biotin, or polyhistidine. The affinity label can be used to affinity purify the peptides or peptide-presenting fusion proteins expressed by host cells using methods known in the art. (See, e.g., Piatibratov et al., supra; Brizzard et al., Biotechniques 16:730-35, 1994; Kloeker & Wadzinski, J. Biol. Chem. 274:5339-47, 1999; incorporated by reference herein) Affinity purified peptides can then be contacted to GPCR-expressing oocytes to screen for activators.

Where the peptides are produced from oligonucleotides, the peptide pool sizes can be estimated based on the complexity of the oligonucleotide library. The term “library complexity” refers to the number of unique clones present in the library. For example, library complexity can be estimated by colony counts of bacteria transformed with oligonucleotide library DNA grown on selective media. Based on the colony counts, the transformed bacteria can be aliquoted so that the aliquots represent the desired pool sizes (e.g., 10,000 or 100,000). Each pool can then be expressed in a separate batch of bacterial host cells. Alternatively, following isolation of pool DNA from bacterial hosts, each pool can be expressed in a non-bacterial host, such as by transformation of yeast with each pool of DNA or transfection of each pool into mammalian host cells.

Combinatorial Chemical Libraries: In another exemplary embodiment, compound pools can be prepared by syntheses of combinatorial chemical libraries (see generally DeWitt et al., Proc. Natl. Acad. Sci. USA 90:6909-13, 1993; International Patent Publication WO 94/08051; Baum, Chem. & Eng. News, 72:20-25, 1994; Burbaum et al., Proc. Natl. Acad. Sci. USA 92:6027-31, 1995; Baldwin et al., J. Am. Chem. Soc. 117:5588-89, 1995; Nestler et al., J. Org. Chem. 59:4723-24, 1994; Borehardt et al., J. Am. Chem. Soc. 116:373-74, 1994; Ohlmeyer et al., Proc. Natl. Acad. Sci. USA 90:10922-26, 1993; and Longman, Windhover's In Vivo The Business & Medicine Report 12:23-31, 1994; all of which are incorporated by reference herein in their entirety.)

The following articles describe methods for selecting starting molecules and/or criteria used in their selection: Martin et al., J. Med. Chem. 38:1431-36, 1995; Domine et al., J. Med. Chem., 37:973-80, 1994; Abraham et al., J. Pharm. Sci. 83:1085-100, 1994; each of which is hereby incorporated by reference in its entirety. Methods of making combinatorial libraries are known in the art, and include the following: U.S. Pat. Nos. 5,958,792; 5,807,683; 6,004,617; 6,077,954 which are incorporated by reference herein.

A “combinatorial library” is a collection of compounds in which the compounds of the collection are composed of one or more types of subunits. The subunits can be selected from natural or unnatural moieties, including dienes, aromatic or polyaromatic compounds, alkanes, cycloalkanes, lactones, dilactones, amino acids, and the like. The compounds of the combinatorial library differ in one or more ways with respect to the number, order, type or types of modifications made to one or more of the subunits comprising the compounds. Alternatively, a combinatorial library may refer to a collection of “core molecules” which vary as to the number, type or position of R groups they contain and/or the identity of molecules composing the core molecule. The collection of compounds is typically generated in a systematic way. Any method of generating a collection of compounds differing from each other in one or more of the ways set forth above can be a combinatorial library.

A combinatorial library can be synthesized on a solid support from one or more solid phase-bound resin starting materials. The library can contain ten (10) or more, typically fifty (50) or more, organic molecules which are different from each other (i.e., ten (10) different molecules and not ten (10) copies of the same molecule). Each of the different molecules (different basic structure and/or different substituents) will be present in an amount such that its presence can be determined by some means (e.g., can be isolated, analyzed, detected with a binding partner or suitable probe). The actual amounts of each different molecule needed so that its presence can be determined can vary due to the procedures used and can change as the technologies for isolation, detection and analysis advance. When the molecules are present in substantially equal molar amounts, an amount, for example, of 100 picomoles or more can be detected. Typical libraries include substantially equal molar amounts of each desired reaction product and typically do not include relatively large or small amounts of any given molecule(s) so that the presence of such molecules dominates or is completely suppressed in any assay.

Combinatorial libraries are generally prepared by derivatizing a starting compound onto a solid-phase support (such as a bead). In general, the solid support has a commercially available resin attached, such as a Rink or Merrifield Resin. After attachment of the starting compound, substituents are attached to the starting compound. For example, an aromatic (e.g., benzene) compound can be bound to a support via a Rink resin. The aromatic ring is reacted simultaneously with a substituent (e.g., an amide). Substituents are added to the starting compound, and can be varied by providing a mixture of reactants to add the substituents. Examples of suitable substituents include, but are not limited to, the following:

(1) hydrocarbon substituents, that is, aliphatic (e.g., alkyl or alkenyl), alicyclic (e.g., cycloalkyl or cycloalkenyl) substituents, aromatic, aliphatic and alicyclic-substituted aromatic nuclei, and the like, as well as cyclic substituents;

(2) substituted hydrocarbon substituents, that is, those substituents containing nonhydrocarbon radicals which do not alter the predominantly hydrocarbon substituent; those skilled in the art will be aware of such radicals (e.g., halo (especially chloro and fluoro), alkoxy, mercapto, alkylmercapto, nitro, nitroso, sulfoxy, and the like);

(3) hetero substituents, that is, substituents that will, while having predominantly hydrocarbyl character, contain other than carbon atoms. Suitable heteroatoms will be apparent to those of ordinary skill in the art and include, for example, sulfur, oxygen, nitrogen, and such substituents as pyridyl, furanyl, thiophenyl, imidazolyl, and the like.

Detection of Electrophysiological Response to Compounds

Following expression of GPCR-encoding RNAs in oocytes, oocytes are exposed to compound pools (or subpools or individual compounds following compound pool subdivision, see infra). Activation of GPCRs can be determined by monitoring oocytes for a GPCR-mediated response. Such a response can be mediated, for example, by a reporter, by electrophysiological response, and the like. Techniques for recording electrophysiological responses in oocytes are known in the art. (See, e.g., Meyerhof et al., Proc. Natl. Acad. Sci. USA 85:714-17, 1988; Wetzel et al., supra; Mulvihill, et al., supra; Getchell et al., supra).

In one exemplary embodiment, an electrophysiological response can be detected using voltage clamp techniques. (See, e.g., Wetzel et al., supra; Getchell et al., supra; Lindqvist, supra.) Oocytes are voltage clamped with, for example, a two-electrode clamp (e.g., GeneClamp 500B, Axon Instruments, Union City Calif.). An appropriate physiological buffer (e.g., ND96, see supra) is perfused across the oocytes and a baseline recording obtained. Following baseline recording, oocytes are perfused with an appropriate recording solution (e.g., hK⁺ (2 mM NaCl₂, 1 mM MgCl₂, 96 mM KCl, 5 mM Na-HEPES, 1 mM CaCl₂) or dilutions of hK⁺ in ND96). Oocyte solutions can, for example, be diluted (e.g., in distilled H₂O) from stock solutions. Concentrated stocks of compounds can be made in an appropriate solution for maintaining solubility of the compounds, typically preserving the ionic and osmotic conditions of the recording solution. Compound pool stocks can then be diluted in recording solutions to desired final concentrations. Oocytes are perfused with recording solution containing final concentrations of compound pools and electrophysiological recordings obtained.

Transmembrane currents are recorded, for example, using two-electrode voltage-clamp techniques with a GeneClamp amplifier. Data is collected and then analyzed accordingly. For example, analog data can be captured by chart recording, as well as converted to digital data using the Digidata analyzer, and recorded and analyzed using pCLAMP8 software (Axon Instruments). Electrodes (e.g., 1.5-2.0 M′ OMEGA) are filled with the appropriate ionic solution (e.g., 3 M KCl). Responses can be recorded at room temperature while the oocyte membrane is voltage-clamped at the desired membrane potential (e.g., −80 mV). Other configurations and equipment that are known in the art can also be used.

Subdivision of Nucleic Acid and Compound Pools to Further Isolate Activators and Identify GPCR Nucleic Acids

Identification of GPCR activators can be achieved, as necessary, through successive subdivisions of activator compound pools. Compounds pools that cause an electrophysiological response when contacted to GPCR-expressing oocytes can be subdivided into two or more subpools of compounds, as described infra, each subpool including a subset of the compounds present in the compound pool. As with the compound pools, these subpools can be contacted to GPCR-expressing oocytes and any electrophysiological response recorded. Activator subpools can be further subdivided to produce a second generation of subpools, which again can be contacted with GPCR-expressing oocytes to identify a second generation of activator subpools. This process can be repeated until at least one subpool of homogeneous compounds produces an electrophysiological response, thereby identifying at least one compound structure as an activator.

Similarly, nucleic acids encoding the activated GPCR(s) corresponding to each identified activator also can be identified by successive subdivisions of nucleic acid pools into subpools, as described infra, each subpool including a subset of the nucleic acid sequences present in the nucleic acid pool. Using the methods described for nucleic acid pools, these subpools can be expressed in oocytes, which can then be contacted with identified activator compounds or, alternatively, activator compound pools or subpools. For each oocyte that demonstrates an electrophysiological response to the activator, the corresponding nucleic acid subpool can be further subdivided to produce a second generation of nucleic acid subpools, which can then be expressed in oocytes that are subsequently contacted with activators. This process can be repeated until at least one subpool of homogeneous compounds confers electrophysiological responsiveness to an activator, thereby identifying at least one nucleic acid sequence encoding the activated GPCR.

Methods for subdivision of compound pools can depend on whether the compounds are synthetically or biologically produced. In the case of synthetically produced compounds, subdivision of pools can be accomplished, for example, by controlling for the synthesis reactions to yield the desired subset of compound structures. For example, in the case of combinatorial chemical libraries, the number of R groups used to produce the compound pool can be reduced (e.g., each subpool could be produced using the same subunits and reactive sites, but with each subpool generated with non-overlapping subsets of the R groups used to generated the original compound pool). Alternatively, if multiple reactive sites are present on the subunits, specific sites can be blocked, thereby limiting substitution of subunits to a subset of reactive sites. Any combination of such techniques can be used accomplish both further subdivisions of subpools and to generate homogeneous compounds.

Similarly, in the case of synthesized peptides, subpools can be prepared by controlling for the possible amino acids at specified positions in the peptide. Certain positions, for example, can remain fixed with other positions variable. Variable positions can also be controlled to include a subset of the possible amino acids.

Subdivision of pools can also be accomplished by any other means that permits tracking of the individual compounds present in each subpool. For example, compound stock dilutions can be aliquoted into 384- or 96-well plates. A single or multiple plates can, for example, represent one compound pool. Subdivision of the pools can, for example, be achieved by preparing subpools from the compounds present in a single plate (if multiple plates represent a pool) or from specified wells of a single plate (if a single plate represents a pool). Such alternative methods for subdivision of compound pools are typically useful for non-combinatorial chemical libraries (e.g., natural products libraries).

Compound pools that are produced biologically from nucleic acids (e.g., peptides or peptide-presenting fusion proteins) can be subdivided by subdividing a pool of nucleic acid clones. Methods for subdividing nucleic acid clones from a library or other groups of nucleic acids are known in the art. (See, e.g., Mulvihill et al., supra; See also Sambrook et al., supra; Ausubel et al., supra.)

For example, subdivision of nucleic acids encoding polypeptide compounds can be accomplished by plating clones from a pool-encoding nucleic acid library onto multiple selective plates. The total number of clones that should be plated to include a clone of interest can be determined, for example, by the probability equation N=1n (1−P)/1n (1−f), where P is the desired probability of including the clone of interest, F is the fraction of positive clones in the pool, and N is the number of clones to be plated to provide the given probability. (See, e.g., Mulvihill et al., supra.) The density of clones plated for each selective plate can be varied according to the desired size of subpools (e.g., from a pool of 100,000, nucleic acid clones can be plated at about 10,000 clones per plate and each plate can represent a subpool). Alternatively, two or more plates can be combined to form a subpool (e.g., from a pool of 100,000, nucleic acid clones can be plated at about 1,000 clones per plate and 10 plates combined to form a subpool). Nucleic acid clones from each subpool can be harvested and stocks prepared from a portion. (See, e.g., Sambrook et al., supra; Ausubel et al., supra.) Subpools can be amplified in liquid culture to produce subpools of polypeptide compounds (e.g., peptides or peptide-presenting fusion proteins), which can be affinity purified according to methods discussed supra.

Alternatively, subdivision of nucleic acids encoding polypeptide compounds can be accomplished by replica plating, for example, a master plate of clones using techniques known in the art. (See, e.g., Mulvihill et al., supra; Sambrook et al., supra; Ausubel et al., supra.) After clones from a master plate are transferred to an appropriate substrate (e.g., nylon or nitrocellulose membrane), the substrate can be divided into sections. Each section, for example, can represent a subpool or, alternatively, two or more sections can be combined to form one subpool. Subpool stocks and encoded polypeptides can be prepared by growing cultures from the replica sections.

Similarly, subdivision of GPCR-encoding nucleic acid pools can be achieved by subdividing a pool of nucleic acid clones. (See discussion regarding subdivision of polypeptide-encoding nucleic acids, supra; Mulvihill et al., supra.; Sambrook et al., supra; Ausubel et al., supra.) Following harvest of nucleic acid clones representing a subpool, vector nucleic acids can be prepared from liquid cultures of host cells. These nucleic acids can then be transcribed in vitro to produce RNA for oocyte injections using methods known in the art (see supra).

Alternatively, RNA pools (e.g., poly (A)⁺ RNA isolated from a natural source) can be subdivided using fractionation methods known in the art. (See, e.g., Sambrook et al.; Ausubel et al, supra.) Such methods include, for example, electrophoresis through agarose gels or sedimentation through sucrose gradients. (See, e.g., Sambrook et al.; Ausubel et al., supra.)

The nucleotide sequence of either identified nucleic acids encoding activated GPCRs of unknown function or, in the case of biologically produced polypeptides, nucleic acids encoding identified activators can be determined by sequencing methods known in the art. (See, e.g., Sambrook et al., supra; Ausubel et al., supra.)

The following examples are provided merely as illustrative of various aspects of the invention and should not be construed to limit the invention in any way.

### Example 1

### Cloning of Receptors for Oocyte Expression

Full length GPCR cDNAs were prepared by PCR and cloned into an expression vector designed to produce stable transcripts for injection and expression in oocytes. GPCR genes of known function, and orphan GPCR sequences were identified in GenBank searches. Oligonucleotides with homology to the 5′ and 3′ termini of the coding region were designed for amplification of the genes of interest. Genes were amplified directly from human genomic DNA if the gene sequences contained no introns, or from mRNA by RT-PCR. The amplified cDNA sequences were gel purified, prepared for cloning by restriction digestion, and cloned into a plasmid vector. Three to six clones of each cDNA were selected and sequenced to identify clones with a perfect sequence map to the published GenBank sequence. The following GPCR genes have been cloned for expression in oocytes (GBAccession): CCR1 (XM_{—}003248), CCR3 (NM_{—}001837), CCR8 (XM_{—}041049), CXCR4 (Y14739), CXCR6 (AF007859), XCR1 (L36149), CCR2B (U03905), CX3CR1 (U28934), CCR5 (U54994), CCR4 (AB023888), CXCR1 (XM_{—}050750), CXCR5 (X68149), CCR6 (XM_{—}033839), Histamine H1 (NM_{—}000861), Histamine H2 (NM_{—}022304), Formyl Peptide R (LI 0820), Platlet Activating Factor Receptor (M76674), Dopamine D2R (NM_{—}000795), Calcitonin R1R (I20773), GPR57 (NM_{—}014627), GPR77 (AF317655), GPR45B (AF118670), and GPR63 (AF317654).

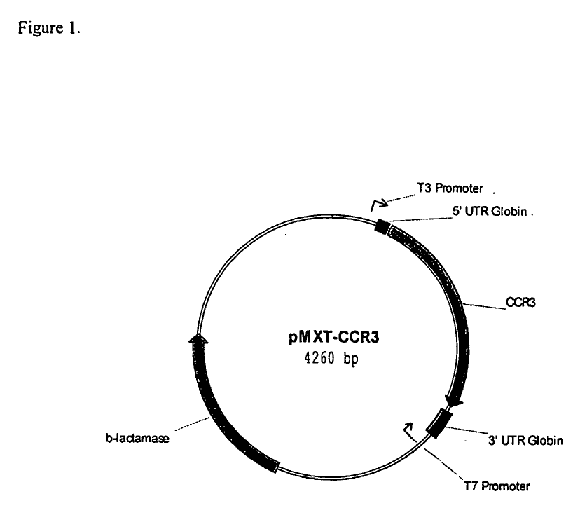

The plasmid vector was engineered for production of RNA transcripts with improved stability and enhanced translational capacity in oocytes. The cDNA inserts were flanked with 5′ and 3′ untranslated regions of the Xenopus globin gene. The construct consisted of a T3 RNA polymerase binding site, the 5′ UTR of the globin gene, the GPCR coding sequence, the 3′ UTR of the globin gene, and a number of unique restriction sites to linearize the construct for transcription as described in Example 2 (FIG. 1).

### Example 2

### Preparation of Nucleic Acid Pools and Polyadenylated RNA for Oocyte Injection

To express exogenous GPCRs encoded by nucleic acid pools, polyadenylated (poly (A)⁺) RNA is prepared for injection in Xenopus oocytes. Messenger RNA for oocyte injection can be prepared from a number of sources including for example; (i) purified polyadenylated RNA from various tissues is available from commercial suppliers including Clontech and InVitrogen. (ii) poly (A)⁺ RNA can be prepared from tissues, cells, cell lines and the like and (iii) RNA can be isolated using common molecular biology techniques, and poly (A)⁺ RNA is purified by affinity to oligo d(T).

Alternatively, a cDNA library can be prepared from poly (A)+ RNA using standard molecular biology techniques. The cDNA is cloned into a vector in which the cDNA insert is flanked at the 5′ side by an RNA polymerase binding site to allow for transcription of message and a restriction endonuclease cleavage site at the 3′ flank to linearize the DNA sequence. Individual clones or library pools are transcribed in vitro using the mMessage mMachine™ system (Ambion) to produce capped transcripts for oocyte injection.

A third alternative involves the isolation of specific cDNA clones for individual GPCR genes by standard molecular biology methods. In this approach, GPCR sequences are identified from a number of sources including publications, or representation in public or private databases. Sequences encoding GPCRs of unknown function are identified based on sequence similarity to known GPCRs. The cDNA sequences are cloned by standard methods, including polymerase chain amplification using oligonucleotides based on the gene sequences, into a vector containing a 5′ RNA polymerase binding site, and a 3′ endonuclease site as described above. The plasmid is utilized as a template for messenger RNA synthesis. Alternatively, the PCR product could be used directly for messenger RNA synthesis. As described above, individual clones or clone pools with up to ˜1000 unique GPCR genes can be transcribed and injected in an individual oocyte.

### Example 3

### Preparation of Compound Pools from Peptide Libraries

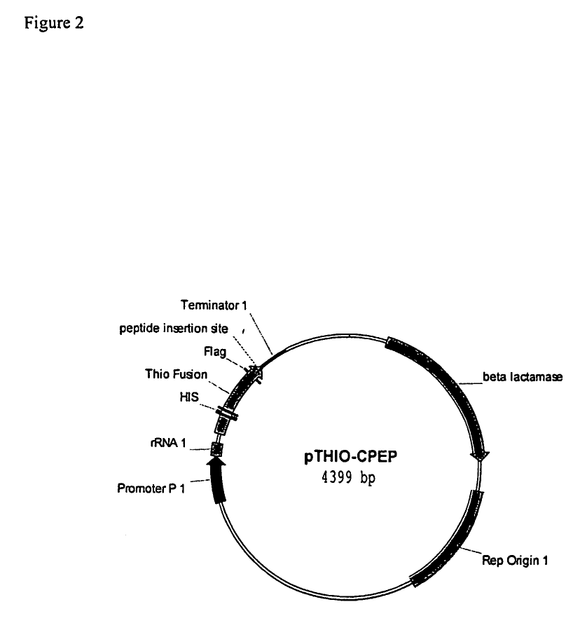

Random peptide libraries were produced by transcription and translation of a library of DNA sequences cloned as a fusion to a scaffold protein. The bacterial thioredoxin gene sequence was utilized by incorporating restriction endonuclease cleavage sites for cloning at the amino terminus, within the active loop or at the carboxy terminus. (See FIG. 2.) Completely random or third position biased oligonucleotides with nonvariant endonuclease sequences for cloning were synthesized, and cloned into the scaffold protein gene sequence at the carboxy terminal position. The scaffold protein contains a 6 residue histidine sequence in the active site for purification by Cobalt affinity chromatography. Ligated DNA was transformed into bacterial cells and grown and selected on ampicillin media. From colony counts, library complexity was estimated to be about 5×10⁹ unique clones.

Based on colony counts, library pool sizes were estimated, and aliquots of transformed bacteria representing various pool sizes (e.g., 10, 100, 1000, 10,000 and 100,000) were grown to high density, diluted in rich media, grown to an OD of 0.5 and the expression of the scaffold/random peptide induced by addition of IPTG. Following 2-4 hours of expression, bacterial cells were pelleted by centrifugation, washed, and lysed with the BPER lysis reagent (Pierce). The lysate was batch purified on Talon resin (Co⁺⁺ affinity chromatography) and buffer exchanged with 0.2×hK, the buffer used for Xenopus patch clamp electrophysiology described above, using a PD10 size exclusion column (Pharmacia). The eluted protein/peptide pool was directly available for analysis in the Xenopus oocyte screening system.

### Example 3

### Electrophysiological Recordings in Oocytes

Xenopus laevis oocytes were used to express nucleic acids encoding GPCRs and to analyze GPCR activation in response to compounds by monitoring changes in membrane potential.

To harvest oocytes, adult female frogs (Xenopus laevis) were anesthetized for about 30 min with 0.2% tricaine before surgery. Sections of one ovary were surgically removed and isolated oocytes were denuded of overlying follicle cells by agitation in 2 mg/ml collagenase (Sigma type IA) in: 82 mM NaCl, 2 mM KCl, 20 mM MgCl₂, 5.0 mM Na-HEPES (pH 7.5) for 1-2 hours. Stage V and VI oocytes were selected.

Following harvest, denuded oocytes were injected with kappa opiate receptor plus GIRK1 and GIRK2 RNA transcribed in vitro using a mMessage mMachine™ kit (Ambion). Cytoplasmic injections of RNA were performed using a glass microelectrode and a NanoJect II injection system (Drummond Scientific Co.). Up to 10 ng of RNA was injected per oocyte in about 50 nl of buffer. In different experimental conditions, injected RNA could include messages encoding GPCR's of interest, G-proteins and ion channels. Oocytes were used for recording 1-2 days after injection and were maintained at 16-19° C. in ND96 (96 mM NaCl, 2 mM KCl, 1 mM MgCl₂, 5 mM Na-HEPES (pH 7.5) 1 mM CaCl₂) plus 2.5 m M Na-pyruvate, 50 μg/ml gentamicin, 5% horse serum.

To record changes in membrane potential in oocytes expressing exogenous GPCRs, ND96 was first perfused across the oocytes for baseline recording. Transmembrane currents were recorded using two-electrode voltage-clamp techniques with a GeneClamp amplifier. Analog data was captured by chart recording, as well as converted to digital form using the Digidata analyzer, and recorded and analyzed using pCLAMP8 software (Axon Instruments). Electrodes (1.5-2.0 M′ OMEGA) were filled with 3 M KCl. Responses were routinely recorded at room temperature while the oocyte membrane was voltage-clamped at −80 mV.

Following baseline recording, oocytes were perfused with hK⁺(2 m M NaCl₂, 1 mM MgCl₂, 96 mM KCl, 5 mM Na-HEPES, 1 mM CaCl₂) or dilutions of hK⁺in ND96. All oocyte solutions were diluted in distilled H₂O from stock solutions. Concentrated stocks of compounds were made and diluted in recording solutions to desired final concentrations. Responses to compounds were routinely recorded at room temperature while the oocyte membrane was voltage-clamped at −80 mV.

### Example 4

### Electrophysiological Recordings in Oocytes Expressing Olfactory Receptors

Olfactory receptors are expressed on the surface of cilia of olfactory neurons in the olfactory epithelium. The olfactory receptors are G-protein coupled seven transmembrane proteins. They are encoded by a family of several hundred to 1000 intronless genes occurring in clusters throughout the genome.

Olfactory receptors are cloned by polymerase chain reaction amplification directly from human genomic DNA using oligonucleotides specific for the amino and carboxy termini. By this approach a large repertoire of individual receptors clones is made available. The receptor genes are cloned into vectors optimized for the in vitro transcription and capping of messenger RNA. RNA transcripts are produced in vitro using the mMessage mMachine™ system (Ambion).

There have been a number of problems reported with obtaining correct expression of olfactory receptors in heterologous systems. However, these receptors have been successfully expressed in Xenopus oocytes. In order to optimize the electrophysiological signal, a number of coupling G-proteins and ion channels are used. Voltage gated potassium channels or the cystic fibrosis transmembrane-conductance regulator (CFTR) are used to obtain a measurable response to receptor ligands. RNAs for these ion channels are produced in vitro as above and co-injected with the olfactory receptor RNA's. Following injection, oocytes are incubated in ND96 for 2 days, and then voltage clamped as described above. For GIRK channels the oocytes are clamped using hK⁺ buffer, while for CFTR analysis, oocytes are clamped using Cl buffer (115 mM NaCl, 2.5 mM KCl, 1.8 mM CaCl₂, and 10 mM HEPES (pH 7.2)). For CFTR analysis, samples of interest are perfused across oocytes in the presence of 1 mM 3-isobutyl-1-methylxanthine and conductance is monitored across a command voltage step from −50 mV to 50 mV (2 second duration). For GIRK analysis, the oocyte is clamped at −80 mV and the responses are measured as membrane conductance over time following ligand perfusion.

### Example 5

### Electrophysiological Recordings in Oocytes Expressing Multiple Receptors

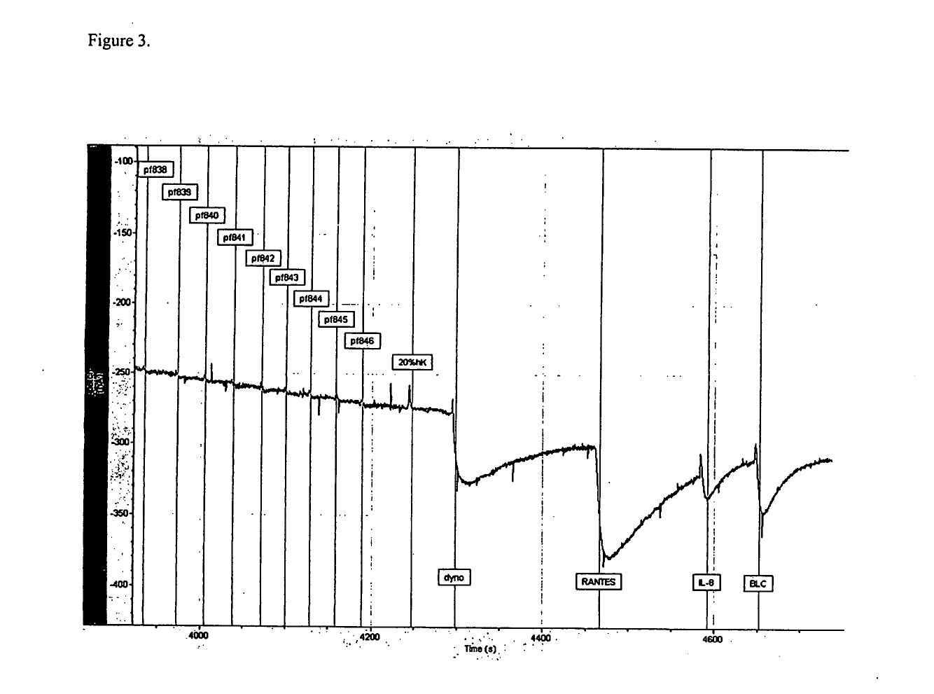

Injection of more than one receptor RNA in oocytes provides for simultaneous analysis of multiple receptors. Receptor RNA's prepared as described in Example 2 can be pooled in combinations of 2 to several hundred receptors, and injected as a receptor pool along with GIRK RNA's. For example, oocytes were injected with a pool of RNAs from 14 receptors (CCR1, CCR3, CCR5, CCR8, CXCR1, CXCR4, CXCR5, CXCR6, XCR1, CX3CR1, GPR45b, GPR57, GPR77, kappa opiate receptor)+GIRK 1&2, incubated at 18° C. for 5 days and voltage clamped in our perfusion system as described in Example 4. Oocytes were perfused with random peptide library pools with an estimated complexity of 10,000 peptides per pool. No detectable response was observed (FIG. 3). Oocytes were subsequently perfused with positive control ligands, and the expected change in current was observed (FIG. 3). With this system it has been established that multiple receptors are expressed when injected simultaneous, and that these receptors are expressed in a functional manner in the oocyte membrane.

The previous examples are provided to illustrate but not to limit the scope of the claimed inventions. Other variants of the inventions will be readily apparent to those of ordinary skill in the art and encompassed by the appended claims. All publications, patents, patent applications and other references cited herein are hereby incorporated by reference.

What is claimed is:

1. A method for identifying an RNA that encodes a G protein-coupled receptor (GPCR) of unknown function in a library of RNAs and for identifying an activator compound, comprising:
simultaneously screening and subdividing the library of RNAs in oocytes and a library of compounds to identify the RNA that encodes the GPCR of unknown function and to identify the activator compound that causes a GPCR-mediated response when contacted with the oocytes expressing the RNA that encodes the GPCR of unknown function.

2. The method of claim 1, wherein the RNA library is complex.

3. The method of claim 1, wherein the compound library is complex.

4. The method of claim 1, wherein the GPCR-mediated response is an electrophysiological response.

5. The method of claim 4, wherein the electrophysiological response is mediated through an endogenous oocyte G protein.

6. The method of claim 1, further comprising introducing a heterologous G protein, or heterologous G protein subunit, into the oocytes, the G protein or G protein subunit effecting the GPCR-mediated response.

7. The method of claim 1, wherein compounds in the compound library are affinity labeled.

8. The method of claim 1, wherein the RNA library comprises poly (A)⁺ mRNAs isolated from human cells or tissues.

9. The method of claim 1, wherein the RNA library comprises RNAs transcribed from cDNAs.

10. The method of claim 1, wherein the RNA library is prepared using subtractive procedures.

11. The method of claim 10, wherein the subtractive procedure is subtractive hybridization.

12. The method of claim 1, wherein the GPCR-mediated response is an increase or decrease in membrane potential.

13. A method for identifying a G protein-coupled receptor (GPCR) of unknown function and an activator compound, comprising:
(a) introducing a heterogeneous RNA pool into oocytes, an RNA in the RNA pool encoding the GPCR of unknown function;
(b) contacting the oocytes with a plurality of pools of compounds;
(c) identifying from the compound pools an activator compound pool that causes a GPCR-mediated electrophysiological response when contacted with the oocytes expressing the pool of heterogeneous RNAs;
(d) subdividing the activator compound pool into compound subpools;
(e) subdividing the RNA pool into RNA subpools;
(f) contacting the compound subpools with oocytes expressing the RNA subpools; and
(g) identifying an activator compound subpool from the compound subpools, and identifying a GPCR RNA subpool, the activator compound subpool causing the GPCR-mediated electrophysiological response when contacted with an oocyte expressing the GPCR RNA subpool.

14. The method of claim 1, wherein the RNA pool is complex.

15. The method of claim 1, wherein the compound pools are complex.

16. The method of claim 13, wherein the GPCR effects the electrophysiological response through an endogenous oocyte G protein.

17. The method of claim 13, further comprising introducing a heterologous G protein, or heterologous G protein subunit, into the oocytes, the G protein or G protein subunit effecting the GPCR-mediated electrophysiological response.

18. The method of claim 13, further comprising repeating steps (d), (e), (f), and (g) to further subdivide the activator compound subpool and GPCR RNA subpool to identify an activator compound that induces the electrophysiological response and to identify a nucleic acid encoding the GPCR activated by the activator compound.

19. The method of claim 18, wherein the GPCR is wild-type.

20. The method of claim 18, wherein the GPCR is a mutant GPCR associated with a disease.

21. The method of claim 13, further comprising:
identifying an activator compound that activates the GPCR receptor; and identifying a nucleic acid that encodes the GPCR receptor.

22. The method of claim 13, wherein the compound pools comprise compound structures that overlap with compound structures of another compound pool.

23. The method of claim 13, wherein the compound pools comprise compound structures that are nonoverlapping with compound structures of other compound pools.

24. The method of claim 13, wherein the compounds in the compound pools are affinity labeled.

25. The method of claim 24, wherein the affinity label is FLAG, V5, myc, biotin, or polyhistidine.

26. The method of claim 13, wherein the RNA pool comprises poly (A)⁺ mRNAs isolated from human cells or tissues.

27. The method of claim 13, wherein the RNA pool comprises RNAs transcribed from cDNAs.

28. The method of claim 13, wherein the RNA encoding the GPCR is identified from a genomic database.

29. The method of claim 28, wherein the GPCR is identified by screening for nucleotide sequences that are substantially similar to a known GPCR.

30. The method of claim 13, wherein the RNA pool is prepared using subtractive procedures.

31. The method of claim 30, wherein the subtractive procedure is subtractive hybridization.

32. The method of claim 13, wherein the electrophysiological response is an increase or decrease in membrane potential.

33. A method for producing a detectable electrophysiological response in an oocyte that is substantially characteristic of activation through a single, homogeneous type of G protein-coupled receptor (GPCR), comprising:
expressing a plurality of different GPCRs of unknown function on an oocyte cell surface;
contacting the oocyte with pools of compounds;
and identifying the electrophysiological response.

34. A method for identifying a G protein-coupled receptor (GPCR) of unknown function and an activator compound, comprising:
(a) providing heterogenous pools of RNAs, at least one of the RNA pools including an RNA encoding the GPCR of unknown function;
(b) providing pools of compounds;
(c) introducing the RNA pools into oocytes to express RNAs contained therein;
(d) contacting the oocytes with the compound pools;
(e) identifying an activator compound pool and a GPCR RNA pool, the activator compound pool causing a GPCR-mediated electrophysiological response when contacted with the oocytes expressing the GPCR RNA pool;
(f) subdividing the activator compound pool to form compound subpools, and subdividing the GPCR RNA pool to form RNA subpools; and
(g) contacting the compound subpools with oocytes expressing the RNA subpools to identify the RNA encoding the GPCR and to identify the activator compound that causes the GPCR-mediated electrophysiological response when the activator compound is contacted with an oocyte expressing the RNA.

35. A method for identifying a G protein-coupled receptor (GPCR) of unknown function and an activator compound, comprising:
(a) introducing a pool of heterogeneous RNAs from normal cells or tissues into first oocytes;
(b) introducing a pool of heterogeneous RNAs from cells or tissues having an altered GPCR-phenotype into second oocytes;
(c) contacting the first and second oocytes with a plurality of pools of compounds;
(d) comparing GPCR-mediated electrophysiological responses in the first and second oocytes to identify a difference in the electrophysiological response between the first and second oocytes;
(e) identifying from the compound pools an activator compound pool, and identifying a GPCR RNA pool from the RNA pools from the cells or tissues having an altered GPCR phenotype, the activator compound pool causing the GPCR-mediated electrophysiological response when contacted to the second oocytes expressing the GPCR RNA pool;
(f) subdividing the activator compound pool into compound subpools;
(g) subdividing the GPCR RNA pool into RNA subpools;
(h) contacting the compound subpools with third oocytes expressing the RNA subpools; and
(i) identifying an activator compound subpool from the compound subpools, and identifying a GPCR RNA subpool, the activator compound subpool causing the GPCR-mediated electrophysiological response when contacted with an oocyte expressing the GPCR RNA subpool.

36. The method of claim 35, further comprising repeating steps (e), (f), (g) and (h) to further subdivide the activator compound subpool and GPCR RNA subpool to identify an activator compound that induces the electrophysiological response and to identify a nucleic acid encoding the GPCR activated by the activator compound.

37. The method of claim 36, wherein the cells or tissues having an altered GPCR phenotype express a mutant GPCR of unknown function.

38. A method for identifying a G protein-coupled receptor (GPCR) of unknown function and an activator compound, comprising:
(a) introducing a heterogeneous RNA pool into oocytes, an RNA in the RNA pool encoding the GPCR of unknown function;
(b) contacting the oocytes with a plurality of pools of random or semi-random peptides;
(c) identifying from the peptide pools an activator peptide pool that causes a GPCR-mediated electrophysiological response when contacted with the oocytes expressing the pool of heterogeneous RNAs;
(d) subdividing the activator peptide pool into peptide subpools;
(e) subdividing the RNA pool into RNA subpools;
(f) contacting the peptide subpools with oocytes expressing the RNA subpools; and
(g) identifying an activator peptide subpool from the peptide subpools, and identifying a GPCR RNA subpool, the activator peptide subpool causing the GPCR-mediated electrophysiological response when contacted with an oocyte expressing the GPCR RNA subpool.

39. The method of claim 38, wherein the RNA pool is complex.

40. The method of claim 38, wherein the peptide pools are complex.

41. The method of claim 38, wherein the GPCR effects the electrophysiological response through an endogenous oocyte G protein.

42. The method of claim 38, further comprising introducing a heterologous G protein, or heterologous G protein subunit, into the oocytes, the G protein or G protein subunit effecting the GPCR-mediated electrophysiological response.

43. The method of claim 38, further comprising repeating steps (d), (e), (f), and (g) to further subdivide the activator peptide subpool and GPCR RNA subpool to identify an activator peptide that induces the electrophysiological response and to identify a nucleic acid encoding the GPCR activated by the activator peptide.

44. The method of claim 43, wherein the GPCR is wild-type.

45. The method of claim 43, wherein the GPCR is a mutant GPCR associated with a disease.

46. The method of claim 38, further comprising:
identifying an activator peptide that activates the GPCR receptor; and identifying a nucleic acid that encodes the GPCR receptor.

47. The method of claim 38, wherein the peptide subpools comprise peptide sequences that overlap with peptide sequences of another peptide subpool.

48. The method of claim 38, wherein the peptide subpools comprise peptide sequences that are nonoverlapping with peptide sequences of other peptide subpools.

49. The method of claim 38, wherein the peptides in the peptide pools are affinity labeled.

50. The method of claim 49, wherein the affinity label is FLAG, V5, myc, biotin, or polyhistidine.

51. The method of claim 38, wherein the RNA pool comprises poly (A)⁺ mRNAs isolated from human cells or tissues.

52. The method of claim 38, wherein the RNA pool comprises RNAs transcribed from cDNAs.

53. The method of claim 38, wherein the RNA pool comprises RNA encoding the GPCR, and the GPCR is identified from a genomic database.

54. The method of claim 53, wherein the GPCR is identified by screening for nucleotide sequences that are substantially similar to a known GPCR.

55. The method of claim 38, wherein the RNA pool is prepared using subtractive procedures.

56. The method of claim 55, wherein the subtractive procedure is subtractive hybridization.

57. The method of claim 38, wherein the electrophysiological response is an increase or decrease in membrane potential.

58. A method for producing a detectable electrophysiological response in an oocyte that is substantially characteristic of activation through a single, homogeneous type of G protein-coupled receptor (GPCR), comprising:
expressing a plurality of different GPCRs of unknown function on an oocyte cell surface;
contacting the oocyte with pools of random or semi-random peptides; and
identifying the electrophysiological response.

59. A method for identifying a G protein-coupled receptor (GPCR) of unknown function and an activator compound, comprising:
(a) providing heterogenous pools of RNAs, at least one of the RNA pools including an RNA encoding the GPCR of unknown function;
(b) providing pools of random or semi-random peptides;
(c) introducing the RNA pools into oocytes to express RNAs contained therein;
(d) contacting the oocytes with the peptide pools;
(e) identifying an activator peptide pool and a GPCR RNA pool, the activator peptide pool causing a GPCR-mediated electrophysiological response when contacted with the oocytes expressing the GPCR RNA pool;
(f) subdividing the activator peptide pool to form peptide subpools, and subdividing the GPCR RNA pool to form RNA subpools; and
(g) contacting the peptide subpools with oocytes expressing the RNA subpools to identify the RNA encoding the GPCR and to identify the activator peptide that causes the GPCR-mediated electrophysiological response when the activator peptide is contacted with an oocyte expressing the RNA.

60. A method for identifying a G protein-coupled receptor (GPCR) of unknown function and an activator compound, comprising:
(a) introducing a pool of heterogeneous RNAs from normal cells or tissues into first oocytes;
(b) introducing a pool of heterogeneous RNAs from cells or tissues having an altered GPCR phenotype into second oocytes;
(c) contacting the first and second oocytes with a plurality of pools of random or semi-random peptides;
(d) comparing GPCR-mediated electrophysiological responses in the first and second oocytes to identify a difference in the electrophysiological response between the first and second oocytes;
(e) identifying from the peptide pools an activator peptide pool, and identifying a GPCR RNA pool from the RNA pools from the cells or tissues having an altered GPCR phenotype, the activator peptide pool causing the GPCR-mediated electrophysiological response when contacted to the second oocytes expressing the GPCR RNA pool;
(f) subdividing the activator peptide pool into peptide subpools;
(g) subdividing the GPCR RNA pool into RNA subpools;
(h) contacting the peptide subpools with third oocytes expressing the RNA subpools; and
(i) identifying an activator peptide subpool from the peptide subpools, and identifying a GPCR RNA subpool, the activator peptide subpool causing the GPCR-mediated electrophysiological response when contacted with an oocyte expressing the GPCR RNA subpool.

61. The method of claim 60, further comprising repeating steps (e), (f), (g) and (h) to further subdivide the activator peptide subpool and GPCR RNA subpool to identify an activator peptide that induces the electrophysiological response and to identify a nucleic acid encoding the GPCR activated by the activator peptide.

62. The method of claim 61, wherein the cells or tissues having an altered GPCR phenotype express a mutant GPCR of unknown function.
